(19) United States
(12) Patent Application Publication
(10) Pub. No.: US 2026/0069560 A1
(43) Pub. Date: Mar. 12, 2026

(54) COMPOSITIONS AND METHODS OF USE

(71) Applicants: UNIVERSITY OF FLORIDA RESEARCH FOUNDATION, INCORPORATED, Gainesville, FL (US); Yale University, New Haven, CT (US)

(72) Inventors: Christian Jobin, Gainesville, FL (US); Rachel Newsome, Gainesville, FL (US); Jason Crawford, New Haven, CT (US); Joonseok Oh, New Haven, CT (US)

(21) Appl. No.: 19/107,637

(22) PCT Filed: Sep. 1, 2023
(86) PCT No.: PCT/US2023/031882

§ 371 Date: Feb. 28, 2025

Related U.S. Application Data
(60) Provisional application No. 63/403,644, filed on Sep. 2, 2022.
(60) Provisional application No. 63/417,833, filed on Oct. 20, 2022.
(60) Provisional application No. 63/432,426, filed on Dec. 14, 2022.

(51) Int. Cl. A61K 31/198; A61K 39/395; A61P 35/00; C07C 237/52

(52) U.S. Cl. CPC A61K 31/198; A61K 39/3955; A61P 35/00; C07C 237/52

(57) ABSTRACT

Aspects of the disclosure relate to compositions and methods for modulating the immune response of a subject. The disclosure is based, in part, on compositions comprising one or more compounds that promotes interferon, e.g., interferon gamma (IFNγ), response in the gut microbiome of a subject and/or enhances the anti-tumor effects of certain immune checkpoint inhibitor (ICI) therapies. In some embodiments, the compositions are useful for treating a subject having cancer.

### CROSS REFERENCE TO RELATED APPLICATIONS AND INCORPORATION BY REFERENCE

This application claims the benefit of U.S. Provisional Patent Application No. 63/432,426, filed Dec. 14, 2022; U.S. Provisional Patent Application No. 63/417,833, filed Oct. 20, 2022; and U.S. Provisional Patent Application No. 63/403,644, filed Sep. 2, 2022, each of which is hereby incorporated by reference in their entireties.

### STATEMENT OF GOVERNMENT SUPPORT

This invention was made with government support under W81XWH-21-1-0547, awarded by Army Medical Research and Development Command. The government has certain rights in the invention.

### FIELD

The invention relates to compositions and methods for modulating the immune response of a subject using same.

### BACKGROUND

Lung cancer remains the leading cause of cancer death among people in the United States. Immune checkpoint inhibitors (ICI) can be highly effective in the treatment of non-small cell lung cancer (NSCLC), but only ˜20-30% of patients experience a complete response. The field of cancer research has been particularly attentive to the interaction between bacteria and therapeutics.

### SUMMARY

The disclosure provides a composition comprising a cell-free isolate obtained from Bacteroides ovatus, wherein the cell free isolate comprises or consists of a first agent having a mass-to-charge ratio (m/z) of 417.2948; a second agent compound having a m/z of 296.25818; a third agent having a m/z of 317.20602; a fourth agent having a m/z of 298.2737; and a fifth agent having a m/z of 430.33078. In some aspects, each agent of the composition is independently selected from a peptide, protein, or small molecule.

In some aspects, the disclosure provides a method for increasing interferon (IFN) secretion in a subject, the method comprising administering to the subject a composition comprising a cell-free isolate obtained from Bacteroides ovatus, wherein the cell free isolate comprises or consists of a first agent having a mass-to-charge ratio (m/z) of 417.2948; a second agent compound having a m/z of 296.25818; a third agent having a m/z of 317.20602; a fourth agent having a m/z of 298.2737; and a fifth agent having a m/z of 430.33078.

In some aspects, the disclosure provides a composition comprising a compound having a structure according to formula (Ia) or a pharmaceutically acceptable salt thereof:

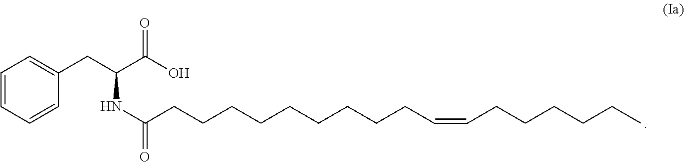 [SMILES: CCCCCC/C=C\CCCCCCCCCC(=O)N[C@@H](Cc1ccccc1)C(=O)O]

In embodiments, the composition further comprises a compound having a structure according to formula (Ib) or a pharmaceutically acceptable salt thereof:

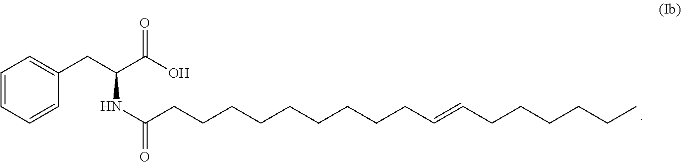 [SMILES: CCCCCC/C=C/CCCCCCCCCC(=O)N[C@@H](Cc1ccccc1)C(=O)O]

In some aspects, the disclosure provides the use of a composition including a compound having a structure according to formula (Ia) or a pharmaceutically acceptable salt thereof and, optionally, a compound having a structure according to formula (Ib) or a pharmaceutically acceptable salt thereof in the manufacture of a medicament for increasing interferon (IFN) secretion in a subject.

In some aspects, the disclosure provides the use of a composition including a compound having a structure according to formula (Ia) or a pharmaceutically acceptable salt thereof and, optionally, a compound having a structure according to formula (Ib) or a pharmaceutically acceptable salt thereof in the manufacture of a medicament for treating cancer in a subject.

In some aspects, the disclosure provides a method for increasing interferon (IFN) secretion in a subject, the method comprising administering to the subject a composition comprising a compound having a structure according to formula (Ia) or a pharmaceutically acceptable salt thereof. In embodiments, the composition further comprises a compound having a structure according to formula (Ib) or a pharmaceutically acceptable salt thereof.

In some aspects, the disclosure provides a method for treating, ameliorating, or preventing cancer in a subject, the method comprising administering to the subject a composition comprising a compound having a structure according to formula (Ia) or a pharmaceutically acceptable salt thereof. In embodiments, the composition further comprises a compound having a structure according to formula (Ib) or a pharmaceutically acceptable salt thereof.

Additional features and variations of the materials and methods of the disclosure will be apparent to those skilled in the art from the entirety of this application, including the figures and detailed description, and all such features are intended as aspects of the invention. Features of the invention described herein can be re-combined into additional embodiments that also are intended as aspects of the invention, irrespective of whether the combination of features is specified as an aspect or embodiment of the invention. The entire document is intended to be related as a unified disclosure, and it should be understood that all combinations of features described herein (even if described in separate sections) are contemplated, even if the combination of features is not found together in the same sentence, or paragraph, or section of this document. Also, only such limitations which are described herein as critical to the invention should be viewed as such; variations of the invention lacking limitations which have not been described herein as critical are intended as aspects of the invention.

### BRIEF DESCRIPTION OF DRAWINGS

FIGS. 1A-1D show representative data indicating that responder microbiota transplantation decreases tumor growth compared to non-responder colonized mice following immunotherapy treatment. FIG. 1A shows a growth curve of Lewis Lung Carcinoma (LLC)-luc subcutaneous xenograft tumors after human fecal microbiota transplant from responder (n=4) or non-responder (n=6) pooled feces into germ-free mice (n=9/group) treated with anti-PD-1 monoclonal antibody injection. P=0.023 at endpoint (ANOVA). (Tumor volume=y-axis; days post LLC implantation=x-axis; square=R feces; circle=NR feces). FIG. 1B shows a growth curve of LLC-luc subcutaneous xenograft tumors after human fecal microbiota transplant from responder (n=4) or non-responder (n=6) pooled feces into germ-free mice (n=5/group). P>0.05 at endpoint (ANOVA). (Tumor volume=y-axis; days post LLC implantation=x-axis; carat=R feces; triangle=NR feces). FIG. 1C shows the mean±SEM of tumor weight at endpoint for mice treated with anti-PD-1 monoclonal antibody. P=0.033 (Mann Whitney test). FIG. 1D shows the mean±SEM of tumor weight at endpoint for untreated mice. P>0.05 (Mann Whitney test). Weight of tumor in grams is depicted on the y-axis.

FIG. 2 shows representative workflow for high-throughput microbial isolation from feces of responder associated (R-associated) mice. The fecal sample is aliquoted and homogenized in anaerobic MEGA media to a final suspension of 10% (w/v). The fecal sample is then diluted to a theoretical target loading of 0.3 cells/well in addition to 50 μM of resorufin as an anaerobic growth indicator. The diluted sample is then vacuum loaded onto 6,000 nanowell growth chambers, sealed and imaged. Wells likely to contain culturable, single bacterial isolates are identified and sterile transfers are performed from the array into 96 well plates from which glycerol stocks can be prepared, cataloged and stored at −80° C. in sealed gas packs. To identify individual isolates, individual bacterial cultures undergo Biotyper analysis. Those isolates that do not receive an identification from the Biotyper can then have genomic DNA extracted for full length 16S PCR and Sanger sequencing by a reference laboratory. This is an exemplary method of obtaining bacteria described herein.

FIG. 3 shows representative workflow for an assay for immunomodulatory effects (IFN, e.g., IFNγ, secretion) of cell free supernatant from individual bacteria. Isolates are cultured in 96-well deep well plates for 3 days under anaerobic conditions in MEGA media. Cell-free supernatant is obtained by filtering cultures by 0.22 μM syringe filtration. Cell-free supernatant is added to the serum-free culture media of primary mouse splenic CD8+ T cells at a ratio of 1:100, followed by 6 hours of incubation. After incubation, cells are harvested and stained for flow cytometric analysis, which is performed on the BD Fortessa LSR, followed by analysis using FlowJo.

FIG. 4 shows a schematic depicting testing for Responder consortium (“R consortium”) anti-PD-1 mediated anti-tumor effect. Germ free mice were colonized with 1×10⁷ colony forming units (CFU) of R consortium, R feces or non-responder (NR) feces for 2 weeks followed by implantation with 1×10⁶ Lewis Lung Carcinoma (LLC) xenograft tumor cells. Mice then underwent four rounds of anti-PD-1 therapy by intraperitoneal injection, with tumors being measured every three days by manual caliper. At endpoint tumors were harvested for flow cytometric analysis.

FIG. 5 shows representative data indicating the Responder consortium decreases tumor growth compared to non-responder (NR) colonized xenograft tumors after human fecal microbiota transplant from R consortium, R feces, or NR pooled feces into germ-free mice treated with anti-PD-1 monoclonal antibody injection. P=0.0046 between R feces and NR feces and P<0.0001 between R consortium and NR feces at endpoint (ANOVA). (Tumor volume=y-axis; days post LLC implantation=x-axis; circle=R consortium; square=R feces; triangle=NR feces)

FIG. 6 shows a schematic depicting testing the effect of anti-IFNγ depletion on R consortium anti-PD-1 mediated anti-tumor effect. Germ-free mice were colonized with 1×10⁷ CFU of R consortium or NR feces for 2 weeks followed by implantation with 1×10⁶ Lewis Lung Carcinoma (LLC) xenograft tumor cells. Mice then underwent four rounds of anti-PD-1 therapy with or without every other day anti-IFNγ therapy, both by intraperitoneal injection, with tumors being measured every three days by manual caliper. At endpoint tumors were harvested for flow cytometric analysis.

FIG. 7 shows representative data indicating in vivo depletion of IFNγ abrogates beneficial effect of responder consortium on anti-PD-1. Growth curve of LLC-luc subcutaneous xenograft tumors after human fecal microbiota transplant from Responder consortium or non-responder pooled feces into germ-free mice treated with anti-PD-1 or combination anti-PD-1/anti-IFNγ monoclonal antibody injection. P=0.041 between R consortium and NR feces and P=0.012 between R consortium and R consortium with anti-IFNγ depletion (ANOVA). No significance between NR feces and NR feces with anti-IFNγ depletion. (Tumor volume=y-axis; days post LLC implantation=x-axis; * anti-IFN; circle=R consortium anti-PD-1; square=R consortium anti-PD-1 and anti-IFNγ; triangle=NR feces anti-PD-1; X=NR feces anti-PD-1 and anti-IFNγ)

FIG. 8 shows a representative process for bioassay-guided fractionation, liquid chromatography, and HPLC for identification of active sub-fractions from cell-free isolates obtained from six-consort bacteria.

FIG. 9 shows representative data indicating compounds produced by Bacteroides ovatus having an active component induce IFNγ production by primary CD8+ T cells. M2H3 F6 sub-fraction F11 (B6 F11) demonstrated the highest level of IFNγ stimulation. Percent of total CD8+ T cells is depicted on the y-axis, and fraction numbers are provided on the x-axis.

FIG. 10A-10B shows representative data indicating that the six (6)-consort enhances anti-PD-1 treatment in vivo through IFNγ. Germ-free LLC tumor-bearing mice were colonized with six-consort or the pooled NR patient samples (10⁷ CFU) and treated with anti-PD-1 or a combination of anti-PD-1/anti-IFNγ antibodies. Six-consort mice treated with only anti-PD-1 showed increased serum IFNγ concentration (FIG. 10A) and tumor infiltrating cytotoxic IFNγ+CD8+ T cells (FIG. 10B) compared to IFNγ-depleted six-consort mice. FIG. 10A shows mean SEM of serum IFNγ concentration in pg/mL 20 days post treatment. P values calculated by Mann-Whitney U test. FIG. 10B shows mean±SEM of intratumoral IFNγ+CD8+ frequency of resected subcutaneous allograft tumors from human microbiota colonized non-responder mice with or without anti-IFNγ depletion (n=4 and n=6, respectively) and six-consort mice with or without IFNγ depletion (n=5 and n=6, respectively). P values calculated by Mann-Whitney U test.

FIGS. 11A-11F illustrate that synthetic cis-Bac430 induces dose-dependent stimulation of IFNγ production. Representative results are shown for IFNγ stimulation from primary splenic CD8+ T cells by cis-Bac430 and trans-Bac430 at a concentration range between 0.01-0.25 mM (FIG. 11A) or between 0.01-1 mM (FIG. 11B). P values calculated using Mann-Whitney U test compared to unstimulated negative control and PMA/Ionomycin-stimulated positive control. Representative results are shown for the percent of live CD8+ T cells from the bioassay for IFNγ stimulation by cis-Bac430 and trans-Bac430 at a concentration range between 0.01-0.25 mM (FIG. 11C) or between 0.01-1 mM (FIG. 11D). P values calculated using Mann-Whitney U test compared to unstimulated negative control and PMA/Ionomycin-stimulated positive control. Representative results are shown for mean±SEM of fecal Bac430 concentration in μM normalized to stool weight from SPF, germ free (GF) or six-consort colonized germ free mice 2 weeks post-colonization (FIG. 11E) or for each member of the six-consort following 3 days anaerobic culture (FIG. 11F).

FIGS. 12A-12G illustrate that intratumoral administration of cis-Bac430 in combination with anti-PD-1 therapy reduces tumor volume and enhances systemic anti-tumor immunity. An experimental schematic is shown for testing anti-PD-1 mediated anti-tumor effect in combination with intratumoral injection of cis-Bac430 or DMSO control (FIG. 12A). Representative results are shown for the growth curve of LLC subcutaneous allograft tumors receiving intratumoral injection of either cis-Bac430 (0.043 mg/mouse) or DMSO alone at the same time as anti-PD-1 treatment or saline control (FIG. 12n). Each point represents tumor volume mean±SEM. At day 20, P values are calculated by Mann-Whitney U test. Representative results are shown for the mean±SEM of tumor weight at the endpoint for mice treated with an anti-PD-1 monoclonal antibody or saline control that received intratumoral injection of either cis-Bac430 or DMSO (FIG. 12C). P values are calculated by Mann-Whitney U test. Representative results are shown for the mean±SEM of intratumoral CD8+ frequency at endpoint for mice treated with an anti-PD-1 monoclonal antibody or saline control that received intratumoral injection of either cis-Bac430 or DMSO (FIG. 12D). P values are calculated by Mann-Whitney U test. Representative results are shown for the mean±SEM of intratumoral IFNγ+CD8+ frequency at endpoint for mice treated with an anti-PD-1 monoclonal antibody or saline control that received intratumoral injection of either cis-Bac430 or DMSO (FIG. 12E). P values are calculated by Mann-Whitney U test. Representative results are shown for the mean±SEM of splenic CD8+ frequency at endpoint for mice treated with an anti-PD-1 monoclonal antibody or saline control that received intratumoral injection of either cis-Bac430 or DMSO (FIG. 12F). P values are calculated by Mann-Whitney U test. Representative results are shown for the mean±SEM of splenic IFNγ+CD8+ frequency at endpoint for mice treated with an anti-PD-1 monoclonal antibody or saline control that received intratumoral injection of either cis-Bac430 or DMSO (FIG. 12G). P values are calculated by Mann-Whitney U test.

### DETAILED DESCRIPTION

The disclosure relates, in various aspects, to compositions and methods for modulating the immune response of a subject. For example, aspects of the disclosure are based, at least in part, on compositions comprising a compound that promotes interferon, e.g., interferon gamma (IFNγ) response in the gut microbiome of a subject.

As described in International (PCT) Patent Application No. PCT/US23/31871, it has been observed that non-small cell lung cancer (NSCLC) patients that respond (R) to immune checkpoint blockade (ICB) have a different microbial community structure than non-responders (NR) pre-treatment. Pooled R microbiota transplantation into gnotobiotic xenograft mice decreased tumor growth compared to NR colonized mice following anti-PD-1 therapy, and this decrease was associated with enrichment of the Bacteroides genus. International (PCT) Patent Application No. PCT/US23/31871 is herein incorporated by reference in the entirety. As described in the Examples below, bioactive molecules that are effective in reducing tumor growth were isolated and identified from cell-free bacterial compositions (e.g., supernatants).

As described herein, the studies presented herein are directed to the identification of small molecules produced by bacteria using bioactivity-guided fractionation coupled with ultra-performance liquid chromatography quadrupole time-of-flight mass spectrometry (UPLC-QTOF-MS)-based comparative metabolomics analysis. Metabolites from Bacterioides having IFNγ stimulatory ability were isolated and identified. One isolate having a strong effect on IFNγ production was selected and cultured before going through subfractionation using vacuum liquid chromatography. Six fractions were generated and tested for their capacity to stimulate IFNγ production from primary splenic CD8+ T cells. As described in the Examples below, Fraction F6 was identified as the most immunostimulatory while also having no significant reduction in cell viability. This fraction was subsequently fractionated into 12 subfractions. A subsequent round of screening showed that only subfraction 11 (F6.11) displayed potent IFNγ stimulatory ability while maintaining viability of the CD8+ T cells. Potential bioactive small molecule characterization of subfraction 11 (F6.11) was performed and revealed five novel small molecule metabolites that are present solely in subfraction 11.

Accordingly, in some aspects, the disclosure provides a composition comprising a cell-free isolate obtained from Bacteroides ovatus, wherein the cell free isolate comprises or consists of a first agent having a mass-to-charge ratio (m/z) of 417.2948; a second agent compound having a m/z of 296.25818; a third agent having a m/z of 317.20602; a fourth agent having a m/z of 298.2737; and a fifth agent having a m/z of 430.33078. In some aspects, each agent of the composition is independently selected from a peptide, protein, or small molecule. In embodiments, the composition comprises, consists essentially of, or consists of at least one agent selected from the group of a first agent having a mass-to-charge ratio (m/z) of 417.2948; a second agent compound having a m/z of 296.25818; a third agent having a m/z of 317.20602; a fourth agent having a m/z of 298.2737; and a fifth agent having a m/z of 430.33078.

In some aspects, the disclosure provides a method for increasing interferon, e.g., interferon gamma (IFNγ), secretion in a subject, the method comprising administering to the subject a composition comprising a cell-free isolate obtained from Bacteroides ovatus, wherein the cell free isolate comprises or consists of a first agent having a mass-to-charge ratio (m/z) of 417.2948; a second agent compound having a m/z of 296.25818; a third agent having a m/z of 317.20602; a fourth agent having a m/z of 298.2737; and a fifth agent having a m/z of 430.33078. In some aspects, the composition comprises a cell-free isolate obtained from Bacteroides ovatus, wherein the cell free isolate comprises or consists an agent having a m/z of 430.33078.

In some aspects, the disclosure provides a method for treating, ameliorating, or preventing cancer in a subject, the method comprising administering to the subject a composition comprising a cell-free isolate obtained from Bacteroides ovatus, wherein the cell free isolate comprises or consists of a first agent having a mass-to-charge ratio (m/z) of 417.2948; a second agent compound having a m/z of 296.25818; a third agent having a m/z of 317.20602; a fourth agent having a m/z of 298.2737; and a fifth agent having a m/z of 430.33078. In some aspects, the composition comprises, consists essentially of, or consists of at least one agent selected from the group of a first agent having a mass-to-charge ratio (m/z) of 417.2948; a second agent compound having a m/z of 296.25818; a third agent having a m/z of 317.20602; a fourth agent having a m/z of 298.2737; and a fifth agent having a m/z of 430.33078. In some aspects, the composition comprises a cell-free isolate obtained from Bacteroides ovatus, wherein the cell free isolate comprises or consists an agent having a m/z of 430.33078.

The amount of cell-free isolate obtained from Bacteroides ovatus administered to a subject in need thereof is an amount effective to achieve a desired biological effect in a clinically relevant time period.

Of the five compounds identified as being present in the active fraction 11, novel metabolite Bac430 (430.33078 m/z) was fully structurally characterized and was found to be produced in a ratio of about 7:1 of cis to trans configurations of the double bond in the long carbon chain.

Bac430 can have a structure according to formula (Ia):

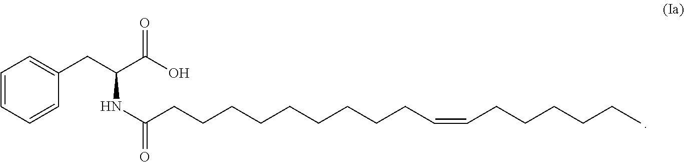 [SMILES: CCCCCC/C=C\CCCCCCCCCC(=O)N[C@@H](Cc1ccccc1)C(=O)O]

The Bac430 isomer having a structure according to formula (Ia) is also referred to herein as cis-Bac430. As described in the Examples below, cis-Bac430 demonstrates dose dependent bioactivity for increasing interferon, e.g., interferon gamma (IFNγ), secretion in a subject. Cis-Bac430 also plays a role in mediating the movement of anti-tumor CD8+ cells from the periphery to the tumor site, thus enhancing the anti-tumor effect of an immune checkpoint inhibitor, such as any of the ICIs described herein.

Bac430 can also have a structure according to formula (Ib):

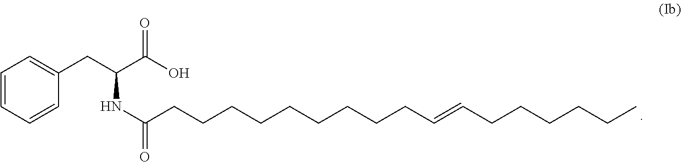 [SMILES: CCCCCC/C=C/CCCCCCCCCC(=O)N[C@@H](Cc1ccccc1)C(=O)O]

The Bac430 isomer having a structure according to formula (Ib) is also referred to herein as trans-Bac430. As described in the Examples below, trans-Bac430, when provided as the sole form of Bac430, does not demonstrate bioactivity for increasing interferon, e.g., interferon gamma (IFNγ), secretion in a subject. Without intending to be bound by theory, it is believed that, in view of the lack of bioactivity for increasing IFNγ secretion in a subject by trans-Bac430 alone, trans-Bac430 when provided as the sole form of Bac430 will not demonstrate bioactivity for enhancing the anti-tumor effect of an immune checkpoint inhibitor, such as any of the ICIs described herein.

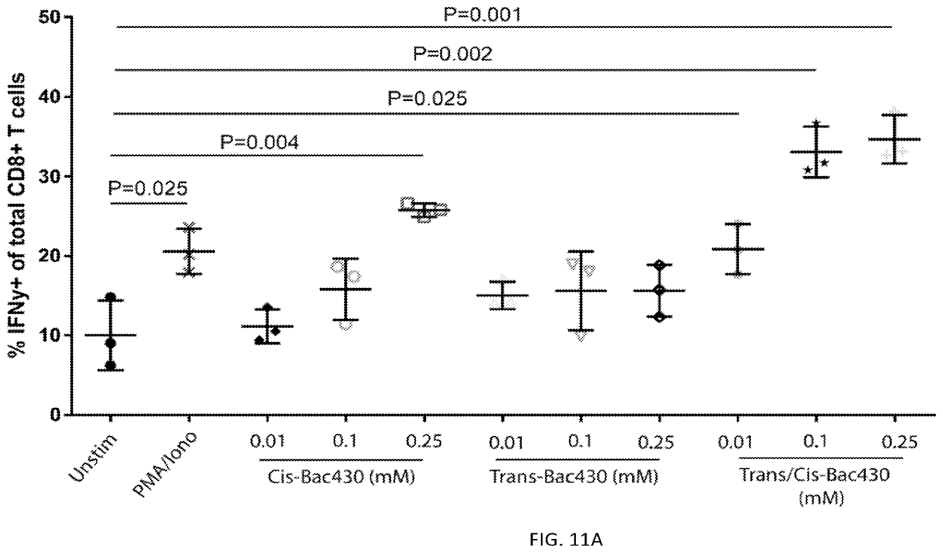

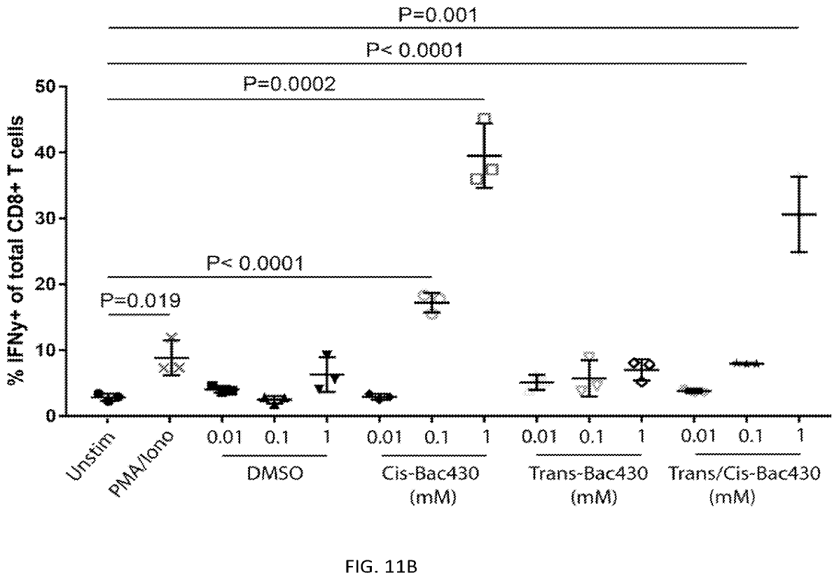

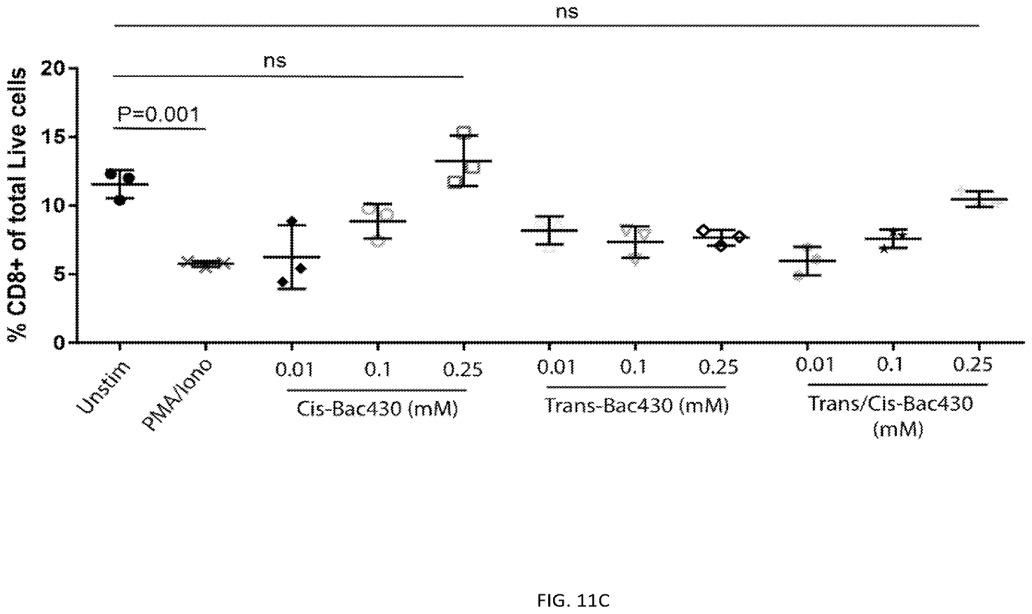

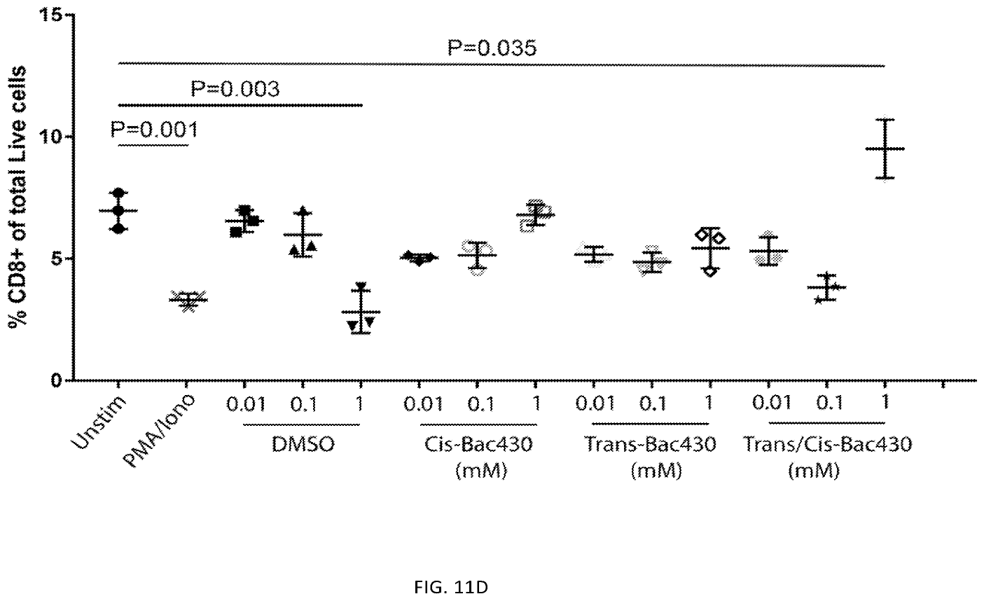

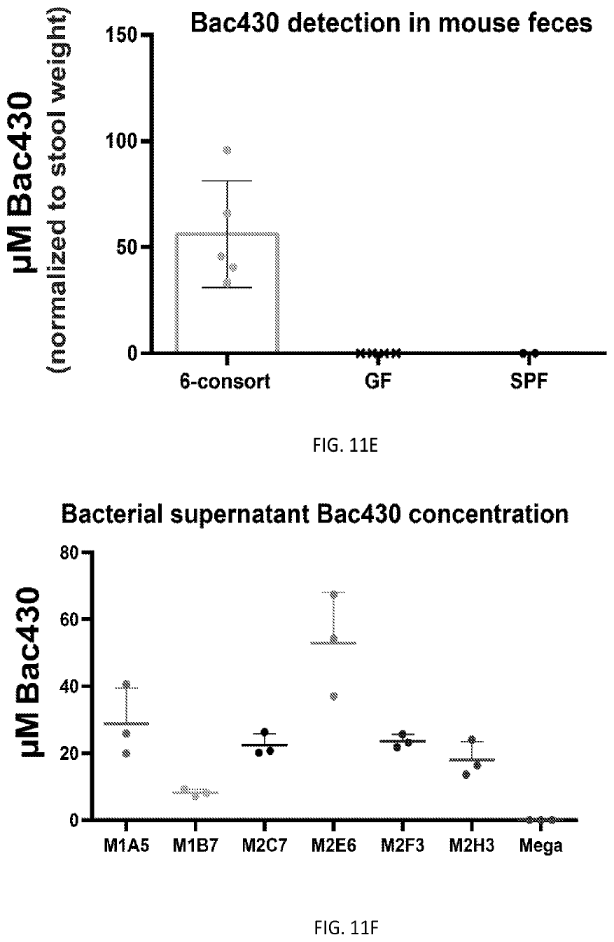

A mixture of compounds having a structure according to formula (Ia) and formula (Ib) is also referred to herein as cis trans-Bac430. Surprisingly, it was found that at some concentrations of Bac430, including trans-Bac430 in combination with cis-Bac430 provided a synergistic effect with respect to the bioactivity for increasing IFNγ secretion. For example, as shown in FIG. 11A, at Bac430 concentrations of 0.10 mM and 0.25 mM, an increase in IFNγ stimulation was demonstrated for the mixture of cis trans-Bac430, relative to the amount of IFNγ stimulation observed for cis-430 alone and trans-430 alone. This result was particularly surprising in view of the inactivity of trans-Bac430 when provided as the sole isomer of Bac430.

Accordingly, in some aspects, the disclosure provides a composition comprising a compound having a structure according to formula (Ia):

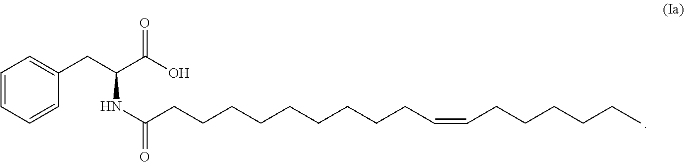 [SMILES: CCCCCC/C=C\CCCCCCCCCC(=O)N[C@@H](Cc1ccccc1)C(=O)O]

In embodiments, the composition further comprises a compound having a structure according to formula (Ib):

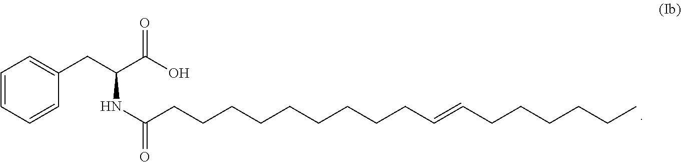 [SMILES: CCCCCC/C=C/CCCCCCCCCC(=O)N[C@@H](Cc1ccccc1)C(=O)O]

(Ib). In general, in a composition including cis-Bac430 and trans-Bac430, cis-Bac430 and trans-Bac430 can be provided in any ratio. In embodiments, the weight ratio of cis-Bac430 to trans-Bac430 can be in a range of 99:1 to 1:99, for example, 95:5, 90:10, 85:15, 80:20, 75:25, 70:30, 65:35, 60:40, 55:45, 50:50, 45:55, 40:60, 35:65, 30:70, 25:75, 20:80, 15:85, 10:90, or 5:95, or in a range of 99:1 to 50:50, 99:1 to 30:70, 99:1 to 15:85 or 99:1 to 5:95. The molar ratio of cis-Bac430 to trans-Bac430 can be, for example, 9:1, 8:1, 7:1, 6:1, 5:1, 4:1, 3:1, 2:1, 1:1, 1:2, 1:3, 1:4, 1:5, 1:6, 1:7, 1:9, or 1:9. In embodiments the molar ratio of cis-Bac430 to trans-Bac430 can be from 9:1 to 1:1, or 7:1 to 1:1, or 5:1 to 1:1, or 3:1 to 1:1.

In embodiments, the composition can be an aqueous composition, wherein the cis-Bac430 and/or cis trans-Bac430 is solubilized in water and/or a water-containing solvent. Although Bac-430 is not intrinsically water-soluble, it was advantageously found that the Bac430 can be pre-dissolved in a suitable solvent, including but not limited to, for example, dimethyl sulfoxide (DMSO), formic acid, alcohols such as ethanol or hexafluoroisopropanol (HFIP), and/or oils such as castor oil or Cremophor (a non-ionic solubilizer and emulsifier that is made by reacting ethylene oxide with castor oil), and then diluted with water to prepare an aqueous composition.

The composition can further comprise a carrier. In general the carrier can be any substance that does not react with the cis-Bac430 or the trans-Bac430. The carrier can be a pharmaceutically acceptable excipient.

The phrase “pharmaceutically acceptable” is employed herein to refer to those ligands, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Compositions described herein can be administered in various forms, depending on the disorder to be treated and the age, condition, and body weight of the patient, as is well known in the art. For example, where the compositions are to be administered orally, they may be formulated as tablets, coated tablets, capsules, granules, powders, suspensions, solutions, slurries, syrups, juices, or emulsions; or for parenteral administration, they may be formulated as injections (intravenous, intramuscular, or subcutaneous), drop infusion preparations, or suppositories. These compositions can be prepared by conventional means in conjunction with the methods described herein, and, if desired, the active ingredient may be mixed with any conventional additive or excipient, such as a binder, a disintegrating agent, a lubricant, a corrigent, a solubilizing agent, a suspension aid, an emulsifying agent, or a coating agent. In embodiments, the composition is an oral composition. In some cases, the oral composition is a tablet, capsule, or suspension. In embodiments, the composition is an injectable composition. For tablets and capsules, an active ingredient, such as the compositions including a compound having a structure according to formula (Ia) and optionally a compound having a structure according to formula (Ib), may be combined with binders, lubricants, disintegrants, and/or colorants. Examples of binders include, but are not limited to, starch, gelatin, natural sugars, natural and synthetic gums such as acacia, tragacanth and sodium oleate, sodium stearate, magnesium stearate, sodium benzoate, sodium acetate, sodium chloride, and the like. Examples of disintegrants include, but are not limited to, starch, methyl cellulose, agar, bentonite, xanthan gum, and the like. Liquid formulations may include carriers such as saline, sterile water, Ringer's solution, buffered saline, dextrose solution, maltodextrin solution, glycerol, or ethanol.

The phrase “pharmaceutically acceptable excipient” as used herein means a pharmaceutically acceptable material, composition, or vehicle, such as a liquid or solid filler, diluent, excipient, solvent or encapsulating material. As used herein the language “pharmaceutically acceptable excipient” includes buffers, sterile water for injection, solvents, dispersion media, coatings, antibacterial and antifungal agents, isotonic and absorption delaying agents, and the like, compatible with pharmaceutical administration. Each excipient must be “acceptable” in the sense of being compatible with the other ingredients of the composition and not injurious to the patient. Some examples of materials which can serve as pharmaceutically acceptable excipients include: (1) sugars, such as lactose, glucose, and sucrose; (2) starches, such as corn starch, potato starch, and substituted or unsubstituted cyclodextrins (α-, β-, or γ-cyclodextrins); (3) cellulose, and its derivatives, such as sodium carboxymethyl cellulose, ethyl cellulose, cellulose acetate, hydroxypropyl methylcellulose (HPMC), hydroxypropyl methylcellulose acetate succinate (HPMCAS); (4) polymers such as polyvinylpyrrolidone (PVP), polyvinylpyrrolidone-vinyl acetate (PVP/VA); (5) surfactants such as sodium lauryl sulfate, polysorbates (Tween), polyoxyethylene stearates (Myri), polyoxyethylene alkyl ethers (Brij), polyethylene glycol, polyvinyl acetate and polyvinylcaprolactame-based graft copolymer (Soluplus), D-α-tocopheryl polyethylene glycol 1000 succinate (TPGS); (6) gelatin; (7) talc; (8) excipients such as cocoa butter and suppository waxes; (9) lipids such as Captex, Capmul and Cremophore; (10) oils, such as peanut oil, cottonseed oil, safflower oil, sesame oil, olive oil, corn oil, and soybean oil; (11) glycols, such as propylene glycol; (12) polyols, such as glycerin, sorbitol, mannitol, and polyethylene glycol; (13) esters, such as ethyl oleate and ethyl laurate; (14) buffering agents, such as magnesium hydroxide and aluminum hydroxide; (15) alginic acid; (16) pyrogen-free water; (17) isotonic saline; (18) Ringer© solution; (19) ethyl alcohol; (20) phosphate buffer solutions; and (21) other non-toxic compatible substances employed in pharmaceutical compositions. In certain embodiments, pharmaceutical compositions provided herein are non-pyrogenic, i.e., do not induce significant temperature elevations when administered to a patient.

The phrase “pharmaceutically acceptable salt” as used herein refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

In embodiments, the compositions of the disclosure comprise at least one pharmaceutically acceptable excipient and a compound of formula (Ia) or a pharmaceutically acceptable salt thereof. In embodiments, the compositions of the disclosure comprise at least one pharmaceutically acceptable excipient, a compound of formula (Ia) or a pharmaceutically acceptable salt thereof, and a compound of formula (Ib) or a pharmaceutically acceptable salt. In embodiments, the compositions or the disclosure consist essentially of at least one pharmaceutically acceptable excipient and a compound of formula (Ia) or a pharmaceutically acceptable salt thereof. In some embodiments, the compositions of the disclosure consist essentially of at least one pharmaceutically acceptable excipient, a compound of formula (Ia) or a pharmaceutically acceptable salt thereof, and a compound of formula (Ib) or a pharmaceutically acceptable salt. In certain embodiments, the compositions of the disclosure consist of at least one pharmaceutically acceptable excipient and a compound of formula (Ia) or a pharmaceutically acceptable salt thereof. In some embodiments, the compositions of the disclosure consist of at least one pharmaceutically acceptable excipient, a compound of formula (Ia) or a pharmaceutically acceptable salt thereof, and a compound of formula (Ib) or a pharmaceutically acceptable salt.

In some embodiments, the compositions of the disclosure are formulated for administration to a mammal. In some embodiments, the compositions of the disclosure are cell-free. In some embodiments, the compositions of the disclosure do not contain any additional biologically active ingredient, such as but not limited to any additional phenylalanine-based derivative(s). As used herein, “phenylalanine-based derivatives” refers to a substituted phenylalanine and, in particular, compounds having a phenylalanine moiety substituted at the amine or carboxylic acid group. In some embodiments, the compositions of the disclosure do not contain any additional biologically active ingredient that elicits a measurable immunomodulatory effect in a mammal, such as but not limited to increasing or promoting IFNγ secretion in the mammal. In some embodiments, the compositions of the disclosure do not contain any additional biologically active ingredient in an amount sufficient to elicit a measurable immunomodulatory effect in a mammal, such as but not limited to increasing or promoting IFNγ secretion in the mammal.

The compound having a structure according to formula (Ia) can be used to modulate the immune response in a subject in need thereof, for example, increasing interferon (IFN) secretion in a subject. Accordingly, the disclosure provides a method for increasing interferon (IFN) secretion in a subject. The method comprises administering to the subject a composition of the disclosure. The method can include administering a composition comprising a compound having a structure according to formula (Ia). In embodiments, the composition further comprises a compound having a structure according to formula (Ib). Methods for measuring IFN production in a subject are well known in the art. Optionally, the composition of the disclosure mediates at least about a 10%, at least about a 20%, at least about a 30%, at least about a 40%, at least about a 50%, at least about a 60%, at least about a 70%, at least about an 80%, or at least about a 90% increase in IFN production in a subject (e.g., as detected in a biological sample from the subject) in a clinically relevant timeframe. The level of IFN production is compared to, e.g., the level of IFN production observed in the subject prior to administration of the composition or compared to a biologically matched control population which is not administered the composition. In various aspects, after the administration of the composition, interferon, e.g., interferon gamma (IFNγ), secretion is increased in the subject relative to IFNγ secretion before the administration.

In some aspects, the disclosure provides a method for treating, ameliorating, or preventing cancer in a subject, the method comprising administering to the subject a composition of the disclosure, for example a composition comprising a compound having a structure according to formula (Ia). In general, the composition can be any composition described herein, including any pharmaceutically acceptable carrier or excipients. In embodiments, the composition further comprises a compound having a structure according to formula (Ib).

The cancer in some aspects is one selected from the group consisting of acute lymphocytic cancer, acute myeloid leukemia, alveolar rhabdomyosarcoma, bone cancer, brain cancer (e.g., glioma), breast cancer (e.g., triple negative breast cancer), cancer of the anus, anal canal, or anorectum, cancer of the eye, cancer of the intrahepatic bile duct, cancer of the joints, cancer of the head, neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal cancer (e.g., gastrointestinal carcinoid tumor), Hodgkin lymphoma, endometrial or hepatocellular carcinoma, hypopharynx cancer, kidney cancer, larynx cancer, liver cancer, lung cancer (e.g., non-small cell lung cancer, bronchioloalveolar carcinoma), malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer. In particular aspects, the cancer is non-small cell lung cancer (NSCLC). In various aspects, the subject has a solid tumor.

In some embodiments, administration of the cis-Bac430 or the combination of cis trans-Bac430 (or a composition described herein) enhances the anti-tumor effects of an immune checkpoint inhibitor (ICI) therapy, for example anti-PD-1 antibodies. An “immune checkpoint inhibitor” or “ICI” is any agent (e.g., compound or molecule) that that decreases, blocks, inhibits, abrogates or interferes with the function of a protein of an immune checkpoint pathway. Proteins of the immune checkpoint pathway regulate immune responses and, in some instances, prevent T cells from attacking cancer cells. In various aspects, the protein of the immune checkpoint pathway is, for example, CTLA-4, PD-1, PD-L1, PD-L2, B7-H3, B7-H4, TIGIT, VISTA, LAG3, CD112 TIM3, BTLA, or co-stimulatory receptor ICOS, OX40, 41BB, or GITR. In various aspects, the ICI is a small molecule, an inhibitory nucleic acid, or an inhibitor polypeptide. In various aspects, the ICI is an antibody, antigen-binding antibody fragment, or an antibody protein product, that binds to and inhibits the function of the protein of the immune checkpoint pathway. Suitable ICIs which are antibodies, antigen-binding antibody fragments, or an antibody protein products are known in the art and include, but are not limited to, ipilimumab (CTLA-4; Bristol Meyers Squibb), nivolumab (PD-1; Bristol Meyers Squibb), pembrolizumab (PD-1; Merck), atezolizumab (PD-L1; Genentech), avelumab (PD-Li; Merck), and durvalumab (PD-Li; Medimmune) (Wei et al., Cancer Discovery 8: 1069-1086 (2018)). Other examples of ICIs include, but are not limited to, IMP321 (LAG3: Immuntep); BMS-986016 (LAG3; Bristol Meyers Squibb); IPH2101 (KIR; Innate Pharma); tremelimumab (CTLA-4; Medimmune); pidilizumab (PD-1; Medivation); MPDL3280A (PD-Li; Roche); MEDI4736 (PD-Li; AstraZeneca); MSB0010718C (PD-Li; EMD Serono); AUNP12 (PD-1; Aurigene); MGA271 (B7-H3: MacroGenics); and TSR-022 (TIM3; Tesaro).

In various aspects, the present disclosure further provides a method of treating a subject in need thereof by administering a composition of the disclosure and further administering one or more ICI to the subject, such as any of the ICIs, or combinations thereof, described herein. In some embodiments, the composition of the disclosure can be administered to the subject prior to administration of the ICI. In some embodiments, the composition of the disclosure is administered to the subject after administration of the ICI. In some embodiments, the composition of the disclosure can be administered to the subject at about the same time or concurrently with the ICI. In some embodiments, the composition of the disclosure can be co-administered to the subject with the ICI. IN some embodiments, the composition of the disclosure can be co-formulated with the ICI.

In various instances, the one or more ICI comprises a PD-1 inhibitor. “Programmed Death-1” (PD-1), also known as cluster of differentiation 279 (CD279), refers to an immunoinhibitory receptor belonging to the CD28 family. PD-1 is expressed on previously activated T cells in vivo, and binds to two ligands, PD-L1 and PD-L2. The human PD-1 sequence can be found under GenBank Accession No. U64863. For example, the PD-1 inhibitor may bind to and inhibit the function of PD-1, e.g., an anti-PD-1 antibody, antigen binding antibody fragment, or an antibody-like molecule. In various aspects, the PD-1 inhibitor is durvalumab, atezolizumab, or avelumab. In various aspects, the ICI is a PD-L2 inhibitor. For example, the PD-L2 inhibitor binds to and inhibits the function of PD-L2, e.g., an anti-PD-L2 antibody, antigen binding antibody fragment, or an antibody-like molecule. Examples of PD-1 and PD-L1 inhibitors are described in, e.g., U.S. Pat. Nos. 7,488,802; 7,943,743; 8,008,449; 8,168,757; 8,217,149; and PCT Patent Publication Nos. WO03042402, WO2008156712, WO2010089411, WO2010036959, WO2011066342, WO2011159877, WO2011082400, and WO2011161699; which are incorporated by reference herein in their entireties. In embodiments, the composition of the disclosure can be administered with a PD-1 inhibitor. In embodiments, the composition of the disclosure can be administered with a PD-1 inhibitor and a second ICI selected from the ICI described herein.

The term “treat,” as well as words related thereto, do not necessarily imply 100% or complete treatment or remission. Rather, there are varying degrees of treatment of which one of ordinary skill in the art recognizes as having a potential benefit or therapeutic effect. In this respect, the methods of treating a disease of the present disclosure can provide any amount or any level of treatment. Furthermore, the treatment provided by the method may include treatment of one or more conditions or symptoms or signs of the disease being treated. For instance, the treatment method of the disclosure may inhibit one or more symptoms of the disease. Also, the treatment provided by the methods of the present disclosure may encompass slowing the progression of the disease. For example, the methods can treat cancer by virtue of enhancing the T cell activity or an immune response against the cancer, thereby reducing tumor or cancer growth, reducing metastasis of tumor cells, increasing cell death of tumor or cancer cells, and the like. Examples of a therapeutic response include (but are not limited to) one or more of the following improvements in the disease: (1) a reduction in the number of neoplastic cells; (2) an increase in neoplastic cell death; (3) inhibition of neoplastic cell survival; (4) inhibition (i.e., slowing to some extent, preferably halting) of tumor growth or appearance of new lesions; (5) decrease in tumor size or burden; (6) absence of clinically detectable disease, (7) decrease in levels of cancer markers; (8) an increased patient survival rate; and/or (9) some relief from one or more symptoms associated with the disease or condition (e.g., pain). For example, the efficacy of treatment may be determined by detecting of a change in tumor mass and/or volume after treatment. The size of a tumor may be compared to the initial size and dimensions as measured by CT, PET, mammogram, ultrasound, or palpation, as well as by caliper measurement or pathological examination of the tumor after biopsy or surgical resection. Response may be characterized quantitatively using, e.g., percentage change in tumor volume (e.g., the method of the disclosure results in a reduction of tumor volume by at least 10%, at least 20%, at least 30%, at least 40%, at least 50%, at least 60%, at least 70%, at least 80%, or at least 90%). Alternatively, tumor response or cancer response may be characterized in a qualitative fashion like “pathological complete response” (pCR), “clinical complete remission” (cCR), “clinical partial remission” (cPR), “clinical stable disease” (cSD), “clinical progressive disease” (cPD), or other qualitative criteria. In addition, treatment efficacy also can be characterized in terms of responsiveness to other immunotherapy treatment or chemotherapy. In various aspects, the methods of the disclosure further comprise monitoring treatment in the subject.

The subject of the methods described herein is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In some aspects, the mammal is a human, optionally a human suffering from or suspected of suffering from cancer.

Actual dosage levels of the cis-Bac430 or the combination of cis-Bac430 and trans-Bac430 in the compositions of the disclosure may be varied so as to obtain a “therapeutically effective amount,” which is an amount of the active ingredient effective to achieve the desired therapeutic response for a particular patient, composition, and mode of administration, without being toxic to the patient. The amount of cis-Bac430 or cis trans-Bac430 administered to a subject in need thereof is an amount effective to achieve a desired biological effect in a clinically relevant time period. For instance, a dose of cis-Bac430 or cis trans-Bac430 administered to the subject can be on the nano-, micro-, or milli-molar scale. In embodiments, a dose of cis-Bac430 or cis trans-Bac430 administered to the subject can be on the nano- or micro-molar scale. In some embodiments, the cis-Bac430 or cis trans-Bac430 can be administered at a concentration in a range of about 5 to about 100 μM, about 10 to about 50 μM, about 10 to about 30 μM, about 15 to about 25 μM, or about 50-100 μM. The concentration of the cis-Bac430 or the combination of cis-Bac430 and trans-Bac430 in the compositions of the disclosure will vary depending on several factors, including the dosage of the compound to be administered, the pharmacokinetic characteristics of the compounds, and the route of administration. Typical dose ranges can include from about 1 mg/Kg to about 100 mg/Kg of body weight per day, and can be given in divided doses, e.g., with each cycle of anti-PD-1 therapy. The dosage will be a therapeutically effective amount depending on several factors including the overall health of a patient, the composition, and the route of administration.

In general, the compositions of the disclosure can be administered using any suitable route of administration. Routes of administration of the compositions of the disclosure can include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, epidural, intrapleural, intraperitoneal, subcutaneous, intramuscular, intradermal, intra-arterial, intravenous, intrabronchial, inhalation, and topical administration. In embodiments, the administration comprises oral or parenteral administration. In embodiments, the administration comprises oral administration. In embodiments, the administration comprises parenteral administration. In embodiments, the administration comprises intratumoral injection.

Various aspects of the disclosure are provided below:

Aspect 1. A composition comprising a cell-free isolate obtained from Bacteroides ovatus, wherein the cell free isolate comprises: (i) a first agent having a mass-to-charge ratio (m/z) of 417.2948; (ii) a second agent compound having a m/z of 296.25818; (iii) a third agent having a m/z of 317.20602; (iv) a fourth agent having a m/z of 298.2737; and (v) a fifth agent having a m/z of 430.33078.

Aspect 2. The composition of aspect 1, wherein each of the first, second, third, fourth, and fifth agents is independently selected from a peptide, protein, or small molecule.

Aspect 3. A method for increasing interferon, e.g., interferon gamma (IFNγ), secretion in a subject, the method comprising administering to the subject the composition of aspect 1 or 2.

Aspect 4. Use in the manufacture of a medicament for increasing interferon, e.g., gamma (IFNγ), secretion in a subject of the composition of aspect 1 or 2.

Aspect 5. Use in the manufacture of a medicament for treating, ameliorating, or preventing cancer of the composition of aspect 1 or 2.

Aspect 6. A composition comprising a compound having a structure according to formula (Ia) or a pharmaceutically acceptable salt thereof:

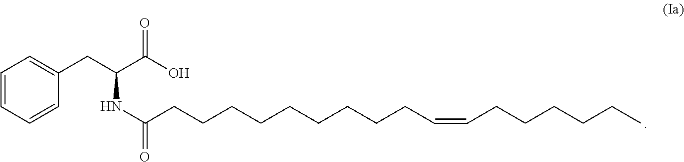 [SMILES: CCCCCC/C=C\CCCCCCCCCC(=O)N[C@@H](Cc1ccccc1)C(=O)O]

Aspect 7. The composition of aspect 6, further comprising a compound having a structure according to formula (Ib) or a pharmaceutically acceptable salt thereof:

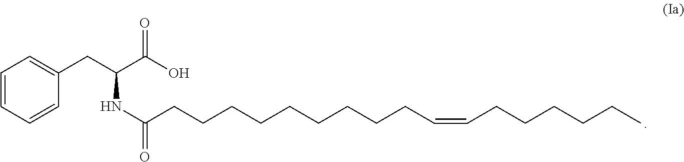 [SMILES: CCCCCC/C=C\CCCCCCCCCC(=O)N[C@@H](Cc1ccccc1)C(=O)O]

Aspect 8. A method for increasing interferon, e.g., interferon gamma (IFNγ) secretion in a subject, the method comprising administering to the subject a composition comprising a compound having a structure according to formula (Ia) or a pharmaceutically acceptable salt thereof.

Aspect 9. The method of aspect 8, wherein the composition further comprises a compound having a structure according to formula (Ib) or a pharmaceutically acceptable salt thereof.

Aspect 10. A method for treating, ameliorating, or preventing cancer in a subject, the method comprising administering to the subject a composition comprising a compound having a structure according to formula (Ia) or a pharmaceutically acceptable salt thereof.

Aspect 11. The method of aspect 10, wherein the composition further comprises a compound having a structure according to formula (Ib) or a pharmaceutically acceptable salt thereof.

### EXAMPLES

### Example 1

The impact of bacteria on therapeutics is wide and includes modulation of chemotherapeutic and immunotherapeutic agents' efficacy and toxicity via metabolic and immune-mediated mechanisms. Intestinal microbiota profoundly impact cancer patients' responses to ICI therapy, and microbial phylogeny is a poor predictor of anti-PD-1 anti-tumor response. Rather, microbial gene content may better capture the relationship between bacterial and ICI responsiveness. To study the relationship between intestinal microbiota and anti-PD-1 efficacy, pre-treatment (baseline) stool samples were obtained from 64 stage III/IV non-small cell lung carcinoma (NSCLC) patients undergoing PD-1 therapy. These patients were categorized as “Responders” or “Non-Responders” using RECIST criteria. It was observed that Responder patients have both a different microbial community structure than Non-Responders (P=0.0043), and a different bacterial transcriptome (PC2=0.03).

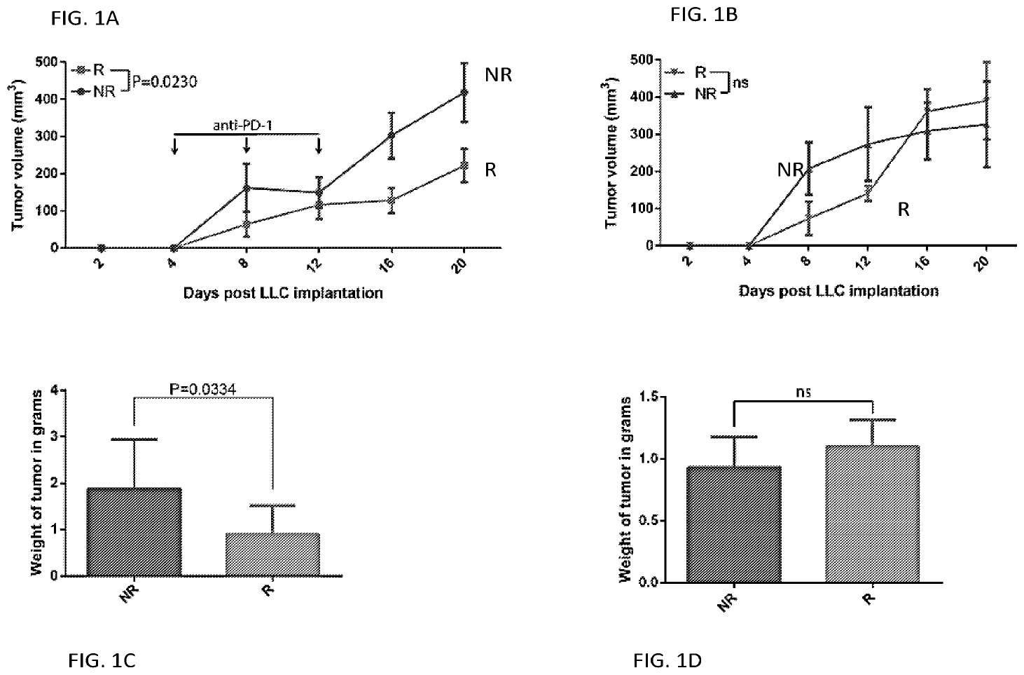

A Lewis Lung Carcinoma (LLC) syngeneic xenograft gnotobiotic mouse model and fecal microbiota transplantation (FMT) approach were used to functionally test anti-PD-1 Responder microbiota. Data indicate a significant decrease in tumor growth and weight in mice colonized with Responder microbiota compared to mice colonized with Non-Responder human feces (FIGS. 1A-1D). In the NSCLC cohort, a higher relative abundance of Ruminococcaceae (genus Ruminococcus and Faecalibacterium) were identified in the feces of PD-1 responding (R) compared to non-responding (NR) patients. Since the syngeneic xenografts were devoid of intratumor bacteria, it is likely that intestinal microbiota exerts synergy with ICI through a systemic, long-distance effect from intestinal biota. It was observed that intestinal microbial-derived metabolites synergize with immune cells to promote anti-PD-1 anti-cancer effect.

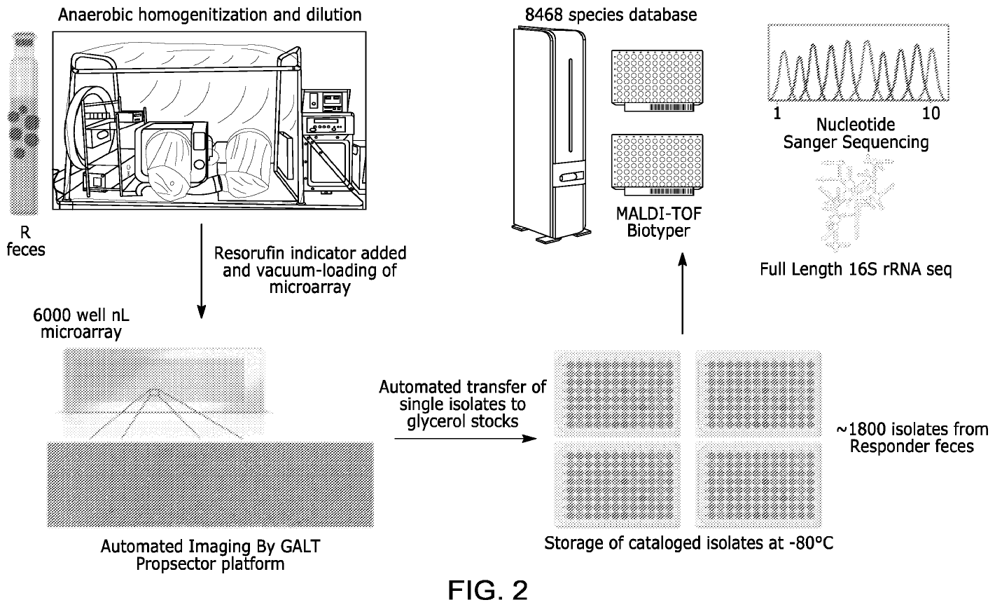

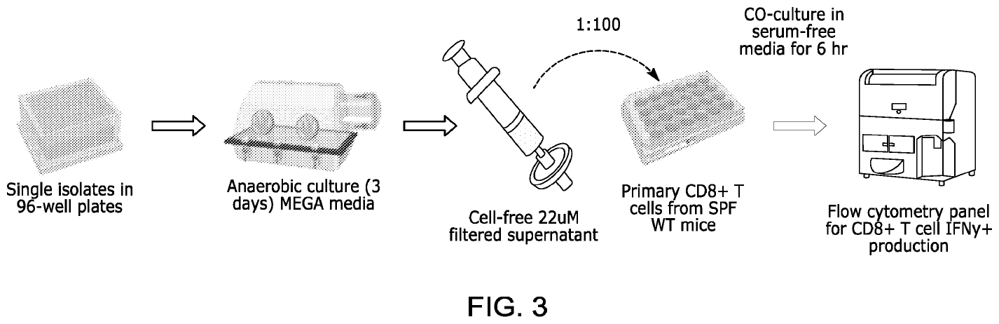

Next, high-throughput microbial isolation from feces of R-associated mice was performed using a GALT Prospector. One hundred and eighty three (183) bacterial strains were identified using MALDI-TOF Biotyper and Sanger sequencing (FIG. 2). The immunomodulatory effect (e.g., interferon (IFN) secretion) of the cell free supernatant of individual cultures of each bacterium was tested on mouse splenic CD8+ T cells (FIG. 3). Following this in vitro screen, six (6) bacterial strains with strong capacity to induce IFN secretion were identified: three (3) strains of Bacteroides ovatus, two (2) strains of Bacteroides intestinalis and one (1) strain of Bacteroides vulgatus. These microorganisms were combined into a consortium designated as six (6)-consort.

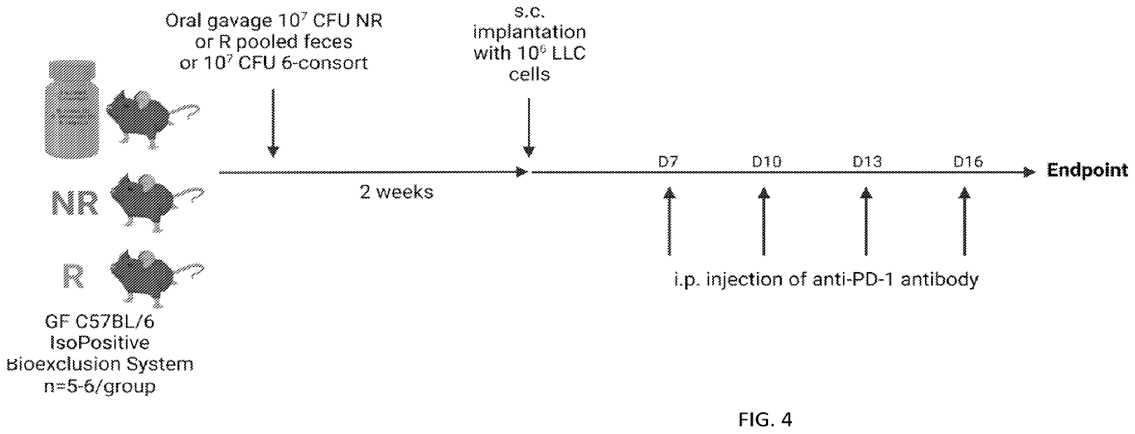

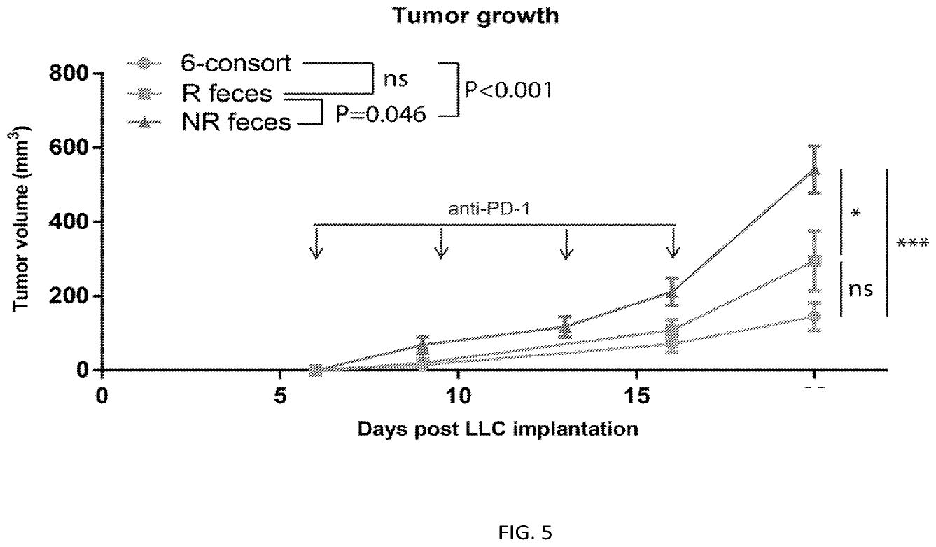

To test the effect of this consortium in anti-PD-1 mediated anti-tumor effect, germ-free mice were administered six-consort (n=8), Responder feces (R-feces) (n=8), or NR feces (n=8) (FIG. 4). Two weeks after colonization, subcutaneous Lewis Lung Carcinoma (LLC) cells were implanted in the mice and once the tumor reached ˜20-30 mm, mice were treated with an anti-PD-1 antibody intraperitonally (i.p.). Four (4) injections were given every three (3) days. A significant decreased tumor growth in mice colonized with six-consort compared to mice colonized with NR-feces was observed (FIG. 5). The anti-tumor effect of the six-consort was as efficient as feces from responding patients (R-feces). These findings indicate that the R Consortium possesses a synergistic effect with anti-PD-1 treatment and may enhance PD-1 responsiveness in patients with NSCLC.

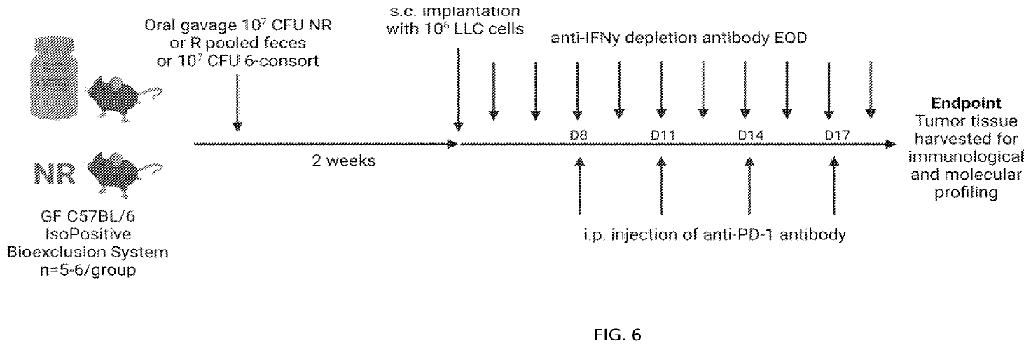

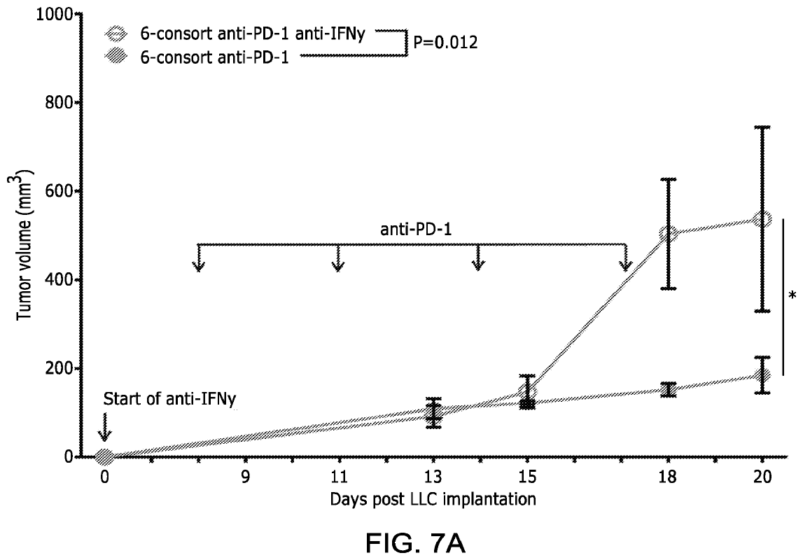

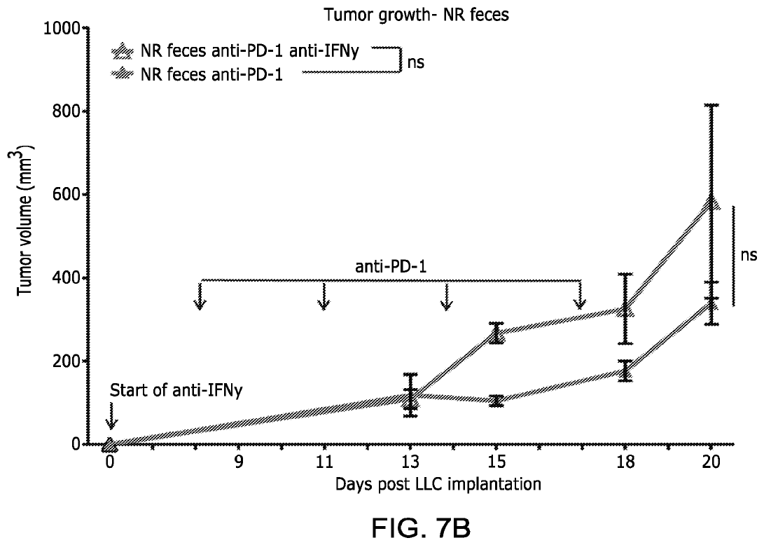

The effects of IFNγ depletion on the anti-tumor effects of six-consort were investigated. FIG. 6 shows a schematic depicting testing the effect of anti-IFNγ depletion on R consortium anti-PD-1 mediated anti-tumor effect. Briefly, germ-free mice were colonized with 1×10⁷ CFU of R consortium or NR feces for 2 weeks followed by implantation with 1×10⁶ Lewis Lung Carcinoma (LLC) xenograft tumor cells. Mice then underwent four rounds of anti-PD-1 therapy with or without every other day anti-IFNγ therapy, both by intraperitoneal injection, with tumors being measured every three days by manual caliper. At endpoint, tumors were harvested for flow cytometric analysis. Data indicate in vivo depletion of IFNγ abrogates the beneficial effect of responder consortium on anti-PD-1. FIG. 7 shows a growth curve of LLC-luc subcutaneous xenograft tumors after human fecal microbiota transplant from Responder Consortium (RC) or Non-Responder (NR) pooled feces into germ-free mice treated with anti-PD-1 or combination anti-PD-1/anti-IFNγ monoclonal antibody injection. No significant difference between NR feces and NR feces with anti-IFNγ depletion was observed.

Previous reports suggested that the presence of bacteria in solid tumors could drive anti-tumor immunity, a process that could alter tumor immune environment and anti-PD-1 efficacy. Given this possibility, tumors were tested to determine if bacteria translocated to the tumor site. The bacterial 16S rRNA gene was not detected in tumors from humanized R and NR mice as assayed by PCR and qPCR, whereas a clear signal was observed in the feces of these mice. Thus, the responder biota mediates their synergistic effect through a long-distance mechanism.

### Example 2

Non-small cell lung cancer (NSCLC) patients that respond (R) to immune checkpoint blockade (ICB) have a different microbial community structure than non-responders (NR) pre-treatment. Example 1 describes that pooled R microbiota transplantation into gnotobiotic xenograft mice decreased tumor growth compared to NR colonized mice following anti-PD-1 therapy, and this decrease is associated with enrichment of the Bacteroides genus. This example describes isolation and identification of cell-free bacterial compositions (e.g., supernatants) containing bioactive molecules that are effective in reducing tumor growth.

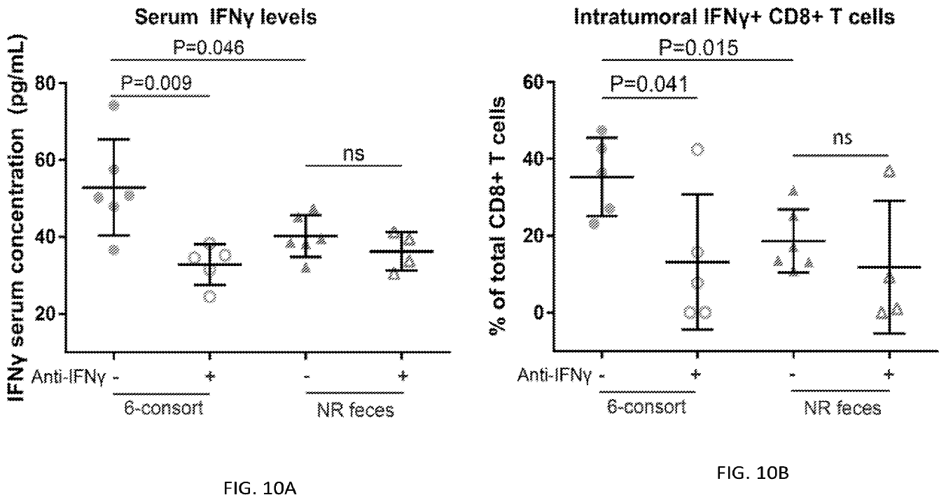

Feces collected from R mice were used as source material for high-throughput microbial isolation performed with the GALT Prospector technology. Bacterial identification was performed using MALDI-TOF Biotyper and Sanger sequencing. The cell free supernatants and <3 kDa small molecules of 183 Bacteroides isolates were screened for their ability to stimulate IFNγ production via a bioassay using primary CD8+ T cells and flow cytometric analysis. A consortium composed of six IFNγ-stimulating isolates or NR feces was transplanted into a gnotobiotic mouse model of lung cancer and treated with anti-PD-1, with or without anti-IFNγ monoclonal antibody depletion. Tumors were harvested at endpoint for flow cytometric analysis, and blood serum for IFNγ ELISA. Six-consort mice treated with only anti-PD-1 showed increased serum IFNγ concentration (FIG. 10A) and tumor infiltrating cytotoxic IFNγ+CD8+ T cells (FIG. 10B) compared to IFNγ-depleted six-consort mice. Vacuum and high-performance liquid chromatography were used to identify fractions from the cell free supernatant of a single stimulating Bacteroides isolate which stimulate IFNγ production.

Six hundred and seventy nine (679) isolates from 30 unique species were cultured and identified. The cell free supernatant from six out of the 183 Bacteroides isolates stimulated IFNγ production from primary CD8+ T cells. Small molecules from the combination of the six stimulatory isolates' supernatant significantly induced IFNγ production in CD8+ T cells compared to six taxonomy-matched non-stimulatory isolates (P=0.039). A defined consortium composed of the six stimulatory isolates (six-consort) was able to colonize germ free mice, and decreased tumor growth compared to NR feces-colonized mice (P=0.041). IFNγ depletion of anti-PD-1-treated six-consort mice significantly increased tumor growth (P=0.012) compared to non-depleted mice. Intratumor IFNγ+CD8+ T cell frequency and circulating serum IFNγ was elevated only in six-consort tumors.

A microbial consortium engineered from R patients' feces synergize with anti-PD-1 therapy to reduce lung cancer growth through an IFNγ-dependent mechanism which may be mediated by small molecule metabolites.

### Example 3

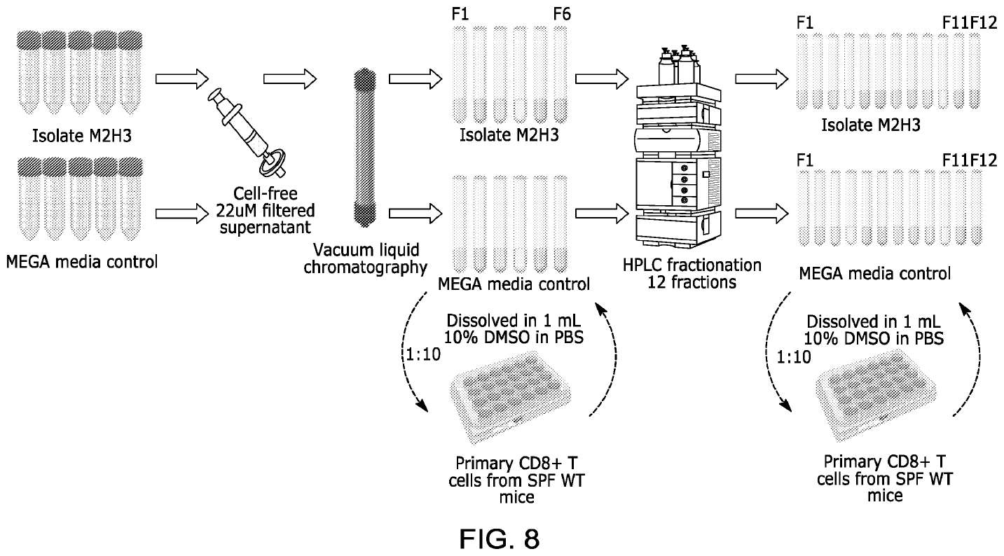

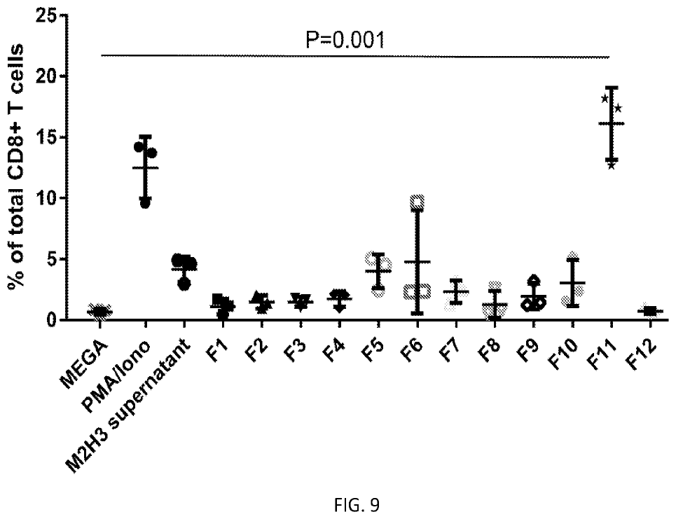

This example describes bioassay-guided fractionation of metabolites collected from the bacteria described herein, revealing five (5) novel small molecules metabolites produced by Bacteroides. To summarize, small molecule characterization was performed using LC-MS/MS and computation pipelines for structural prediction. To identify specific bioactive small molecules produced by an immunostimulatory Bacteroides ovatus isolate, bioassay guided fractionation along with liquid chromatography and HPLC were used to identify an active sub-fraction (FIG. 8). In particular, Bacteroides isolate M2H3 was selected based on its strong effect on IFNγ production. Isolate M2H3 was cultured in MEGA media anaerobically for 3 days, and the culture supernatant was filtered through a 0.22 μm membrane before going through subfractionation using reverse phase (RP) vacuum liquid chromatography. This step generated six fractions (F1-F6) that were tested for their capacity to stimulate IFNγ production from primary splenic CD8+ T cells. Fraction F6 was identified as the most immunostimulatory while also having no significant reduction in cell viability. This fraction was subsequently fractionated into 12 subfractions (F6.1-F6.12) using semi-preparative high-performance lipid chromatography (HPLC). A subsequent round of screening showed that only subfraction 11 displayed potent IFNγ stimulatory ability while maintaining viability of the CD8+ T cells. Using large scale culture along with LCMS/MS and NMR analysis, five (5) compounds present solely in fraction 11 were identified as having mass-to-charge ratios (m/z) of 417.2948, 296.25818, 317.20602, 298.2737 and 430.33078. The compounds were produced by Bacteroides ovatus. This approach identified fraction eleven (11) as having an active component inducing IFNγ production by primary CD8+ T cells (FIG. 9).

The active (i.e., F6 and F6.11) and inactive fractions were analyzed by UPLC-QTOF-MS, which led to the discovery of two potentially isomeric candidate ion features in the active fraction (m/z 430.3317; major:minor=7:1). Targeted tandem MS analysis followed by database searches (as described in Nature Methods 2020, 17, 905-908, herein incorporated by reference in the entirety) was used to identify the chemical entities as N-acyl amides, featuring a phenylalanine residue coupled with a monounsaturated C18 fatty acid chain. The configuration of the phenylalanine moiety was established as (S) based upon chemical degradation and chiral functionalization (Marfey's analysis, as described in Anal. Chem. 1997, 69, 5146-5151, herein incorporated by reference in the entirety), and the double bond position of the lipid was established as ω-7 (vaccenic acid) using an olefin cross metathesis approach as described in Angew. Chem. Int. Ed. 2011, 50, 8275-8278, herein incorporated by reference in the entirety. The cis and trans geometric isomers of the proposed N-acyl amides, cis-Bac430 and trans-Bac430, respectively, were synthesized and confirmed to be the major and minor metabolites in the active fraction by comparative UPLC-QTOF-MS and co-injection studies.

Thus, novel metabolite Bac430 (m/z) was fully structurally characterized by synthesis and NMR (comparisons of synthetic versus natural metabolites), chemical degradation and chiral functionalization (Marfey's analysis of the natural metabolite to establish the (S) configuration of the amino acid), and an olefin cross metathesis approach to establish the ω-7 position of the double bond in the lipid chain, and was found to be produced in a ratio of about 7:1 of cis to trans configurations of the double bond in the long carbon chain. Bac430 was selected for testing for stimulatory activity because of its fully characterized structure.

### Example 4

This example describes the preparation of synthetic cis-Bac430 and trans-Bac430. To establish the structures of cis-Bac430 and trans-Bac430, L-phenylalanine t-butyl ester was coupled to either cis- or trans-vaccenic acids using the carboxylic acid activating regent PyBOP (benzotriazol-1-yloxytripyrrolidinophosphonium hexafluorophosphate). Then the t-butyl protecting group of the amide products was deprotected with trifluoroacetic acid. The two synthetic standards were compared with the B. ovatus-derived active fraction via UPLC-QTOF-MS.

Aqueous solutions of Bac430 were also be prepared. In short, dried compounds are weighed and the exact weights reported. Dimethyl sulfoxide (DMSO) was added to each compound relative to their molecular weight and total mass to reach a stock concentration of 10 mM. Agitation and heating to 50° C. allowed for total dissolution. Dissolved compounds were aliquoted and frozen at −20° C. until use. For in vitro experiments, appropriate volumes of the 10 mM stock DMSO solutions were thawed and added to RPMI media at 37° C. to achieve working concentrations between 0.01-1 mM for splenocyte simulation. In vivo, mice were injected with undiluted DMSO stock solution.

### Example 5

This example describes the use of Bac430 to stimulate IFNγ production. A synthetic version of both cis- and trans-Bac430 were generated as described in Example 4 to characterize bioactivity using primary splenic CD8+ T cells. Cis-Bac430 alone and the combination of cis trans-Bac430 were shown to robustly stimulate IFNγ from primary murine splenic CD8+ T cells in a dose-dependent manner (FIGS. 11A and 11B).

Additionally, a mild proliferative effect was observed at higher concentrations of cis-Bac430 on CD8+ T cells specifically (FIGS. 11C and 11D). To examine the endogenous production of Bac430 in vivo and the concentration produced by the six-consort, metabolomics analysis was done on feces of germ free, SPF and germ-free mice colonized with the consortium. Germ free mice colonized with the six-consort showed high levels of Bac430 in the feces (between 50-100 μM), whereas this small molecule is undetectable in the feces of germ free or SPF mice, indicating that this is not produced endogenously at detectable level (FIG. 11E). Interestingly, all isolates of the six-consort were found to produce Bac430 in the cell culture supernatant at concentrations around 10-50 μM, with isolate M2H3 only producing around 20 μM (FIG. 11F).

### Example 6

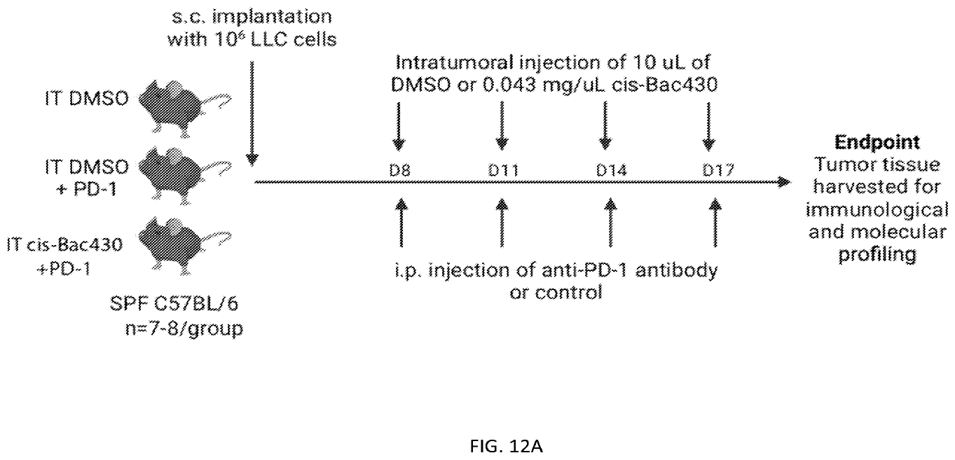

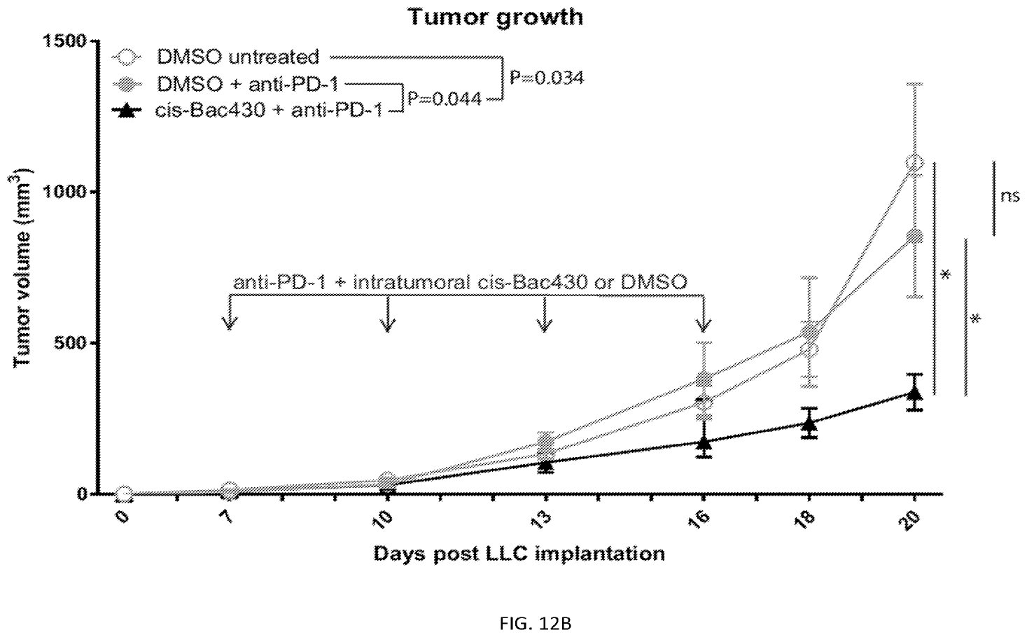

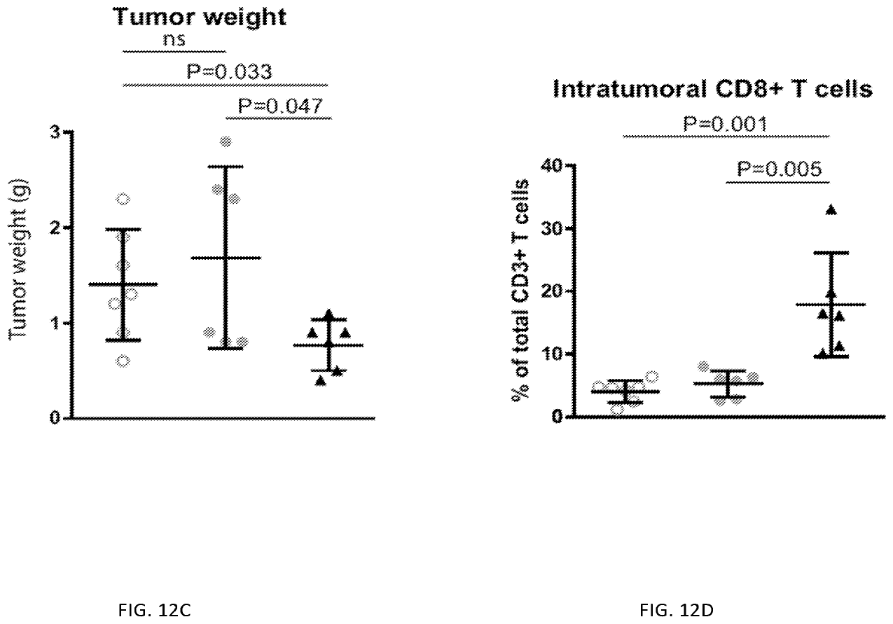

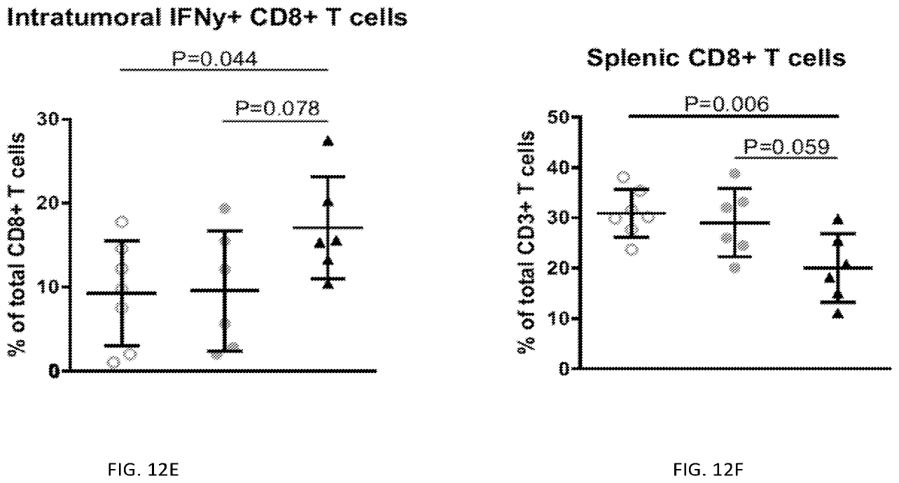

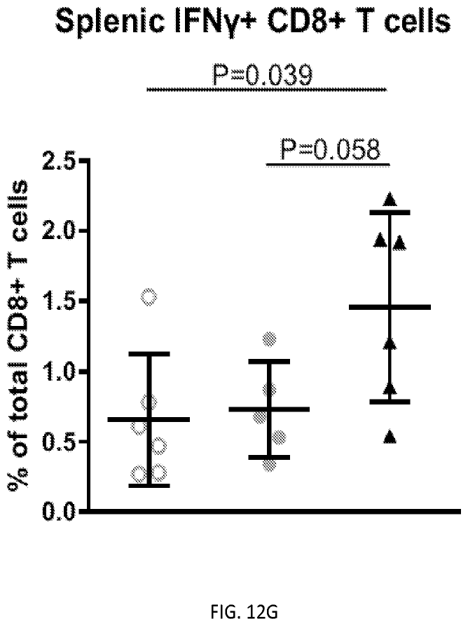

This example demonstrates the use of Bac430 to enhance anti-tumor immunity. Lung tumor-bearing mice were injected intratumorally with a 10 μL volume of either 10 mM (0.043 mg/dose) cis-Bac430 or DMSO on each day of anti-PD-1 therapy (FIG. 12A). Mice receiving intratumoral cis-Bac430 showed significantly smaller tumor volume and weight at endpoint compared to anti-PD-1 or DMSO injection (FIGS. 12B and 12C). Cis-Bac430-treated mice combined with anti-PD-1 showed increased frequency of intratumoral CD8+ T cells and a trend toward increased tumor infiltrating cytotoxic IFNγ+CD8+ T cells (FIGS. 12D and 12E). Interestingly, although cis-Bac430 was administered intratumorally, splenic immune profiling showed a trend toward increased IFNγ+CD8+ T cells but a decreased overall CD8+ T cell frequency in mice treated with cis-Bac430, indicating that the movement of anti-tumor CD8+ cells from the periphery to the tumor site was affected by cis-Bac430 treatment (FIGS. 12F and 12G). Taken together, these results indicate that cis-Bac430 is a small molecule metabolite partially responsible for mediating anti-tumor synergy with immune checkpoint inhibition by the Bacteroides genus.

Cis-Bac430 also plays a role in mediating the movement of anti-tumor CD8+ cells from the periphery to the tumor site thus enhancing the anti-tumor effect of anti-PD-1.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context; the terms “a” (or “an”), “one or more,” and “at least one” can be used interchangeably herein. The term “or” should be understood to encompass items in the alternative or together, unless context unambiguously requires otherwise. The term “and/or” should be understood to encompass each item in a list (individually), any combination of items a list, and all items in a list together. The terms “comprising,” “having,” “including,” and “containing” are to be construed as open-ended terms (i.e., meaning “including, but not limited to,”) unless otherwise noted. The disclosure contemplates embodiments described as “comprising” a feature to include embodiments which “consist of” or “consist essentially of” the feature. Other than in the operating examples, or where otherwise indicated, all numbers expressing quantities of ingredients or reaction conditions used herein should be understood as modified in all instances by the term “about” as that term would be interpreted by the person skilled in the relevant art. The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, per the practice in the art. Alternatively, “about” can mean a range of up to 10%, up to 5%, or up to 1% of a given value.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein. In any of the ranges described herein, the endpoints of the range are included in the range. However, the description also contemplates the same ranges in which the lower and/or the higher endpoint is excluded.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

What is claimed:

1. A composition comprising a compound having a structure according to formula (Ia) or a pharmaceutically acceptable salt thereof:

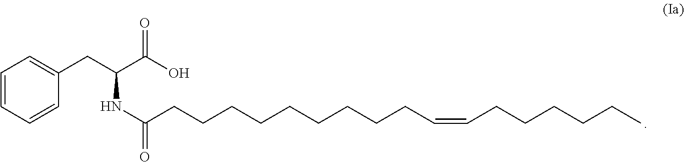 [SMILES: CCCCCC/C=C\CCCCCCCCCC(=O)N[C@@H](Cc1ccccc1)C(=O)O]

2. The composition of claim 1, further comprising a compound having a structure according to formula (Ib) or a pharmaceutically acceptable salt thereof:

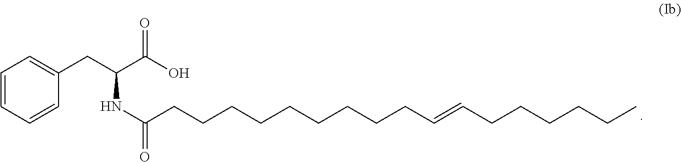 [SMILES: CCCCCC/C=C/CCCCCCCCCC(=O)N[C@@H](Cc1ccccc1)C(=O)O]

3. The composition of claim 2, wherein the compound having a structure according to formula (Ia) and the compound having a structure according to formula (Ib) are provided in a weight ratio in a range of 99:1 to 1:99, for example, 95:5, 90:10, 85:15, 80:20, 75:25, 70:30, 65:35, 60:40, 55:45, 50:50, 45:55, 40:60, 35:65, 30:70, 25:75, 20:80, 15:85, 10:90, or 5:95, or in a range of 99:1 to 50:50, 99:1 to 30:70, 99:1 to 15:85 or 99:1 to 5:95.

4. The composition of any one of claims 1 to 3, wherein the composition is an aqueous composition.

5. The composition of any one of claims 1 to 4, further comprising a pharmaceutically acceptable carrier.

6. Use in the manufacture of a medicament for increasing interferon gamma (IFNγ) secretion in a subject of a composition according to any one of claims 1 to 5.

7. Use in the manufacture of a medicament for treating cancer of a compound according to any one of claims 1 to 5.

8. A method for increasing interferon gamma (IFNγ) secretion in a subject, the method comprising administering to the subject a composition comprising a compound having a structure according to formula (Ia) or a pharmaceutically acceptable salt thereof:

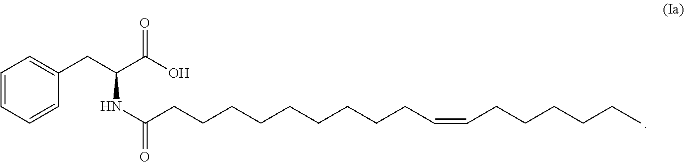 [SMILES: CCCCCC/C=C\CCCCCCCCCC(=O)N[C@@H](Cc1ccccc1)C(=O)O]

9. The composition of claim 8, further comprising a compound having a structure according to formula (Ib) or a pharmaceutically acceptable salt thereof:

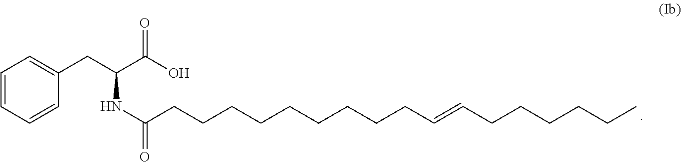 [SMILES: CCCCCC/C=C/CCCCCCCCCC(=O)N[C@@H](Cc1ccccc1)C(=O)O]

10. The method of claim 8 or 9, wherein the subject is a mammal, optionally a human.

11. The method of any one of claims 8 to 10, wherein the administration comprises oral administration.

12. The method of any one of claims 8 to 10, wherein the administration comprises parenteral administration.

13. The method of any one of claims 8 to 12, wherein the composition is an aqueous composition.

14. The method of any one of claims 8 to 13, wherein the subject has or is suspected of having cancer.

15. The method of claim 14, wherein the cancer is non-small cell lung carcinoma (NSCLC).

16. The method of any one of claims 8 to 15, further comprising administering an immune checkpoint inhibitor (ICI) to the subject.

17. The method of claim 16, wherein the ICI comprises an anti-PD-1 antibody.

18. A method for treating, ameliorating, or preventing cancer in a subject, the method comprising administering to the subject a composition comprising a compound having a structure according to formula (Ia) or a pharmaceutically acceptable salt thereof:

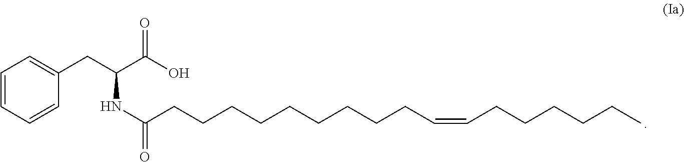 [SMILES: CCCCCC/C=C\CCCCCCCCCC(=O)N[C@@H](Cc1ccccc1)C(=O)O]

19. The composition of claim 18, further comprising a compound having a structure according to formula (Ib) or a pharmaceutically acceptable salt thereof:

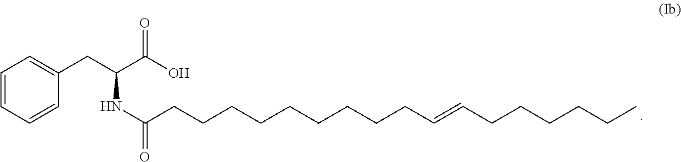 [SMILES: CCCCCC/C=C/CCCCCCCCCC(=O)N[C@@H](Cc1ccccc1)C(=O)O]

20. The method of claim 18 or 19, wherein the subject is a mammal, optionally a human.

21. The method of any one of claims 18 to 20, wherein the administration comprises oral administration.

22. The method of any one of claims 18 to 20, wherein the administration comprises parenteral administration.

23. The method of any one of claims 18 to 22, wherein the composition is an aqueous composition.

24. The method of any one of claims 18 to 23, wherein the cancer is non-small cell lung carcinoma (NSCLC).

25. The method of any one of claims 18 to 24, further comprising administering an immune checkpoint inhibitor (ICI) to the subject.

26. The method of claim 25, wherein the ICI comprises an anti-PD-1 antibody.

27. The method of any one of claims 18 to 26, wherein after the administration of the composition, interferon gamma (IFNγ) secretion is increased in the subject relative to IFNγ secretion before the administration.

28. A composition comprising a cell-free isolate obtained from Bacteroides ovatus, wherein the cell free isolate comprises:
(i) a first agent having a mass-to-charge ratio (m/z) of 417.2948;
(ii) a second agent compound having a m/z of 296.25818;
(iii) a third agent having a m/z of 317.20602;
(iv) a fourth agent having a m/z of 298.2737; and
(v) a fifth agent having a m/z of and 430.33078.

29. The composition of claim 28, wherein each of the first, second, third, fourth, and fifth agents is independently selected from a peptide, protein, or small molecule.

30. A method for increasing interferon gamma (IFNγ) secretion in a subject, the method comprising administering to the subject the composition of claim 28 or 29.

31. A method for treating, ameliorating, or preventing cancer in a subject, the method comprising administering to the subject the composition of any one of claims 28 to 30.

32. The method of claim 30 or 31, wherein the subject is a mammal, optionally a human.

33. The method of any one of claims 30 to 32, wherein the administration comprises oral administration.

34. The method of any one of claims 30 to 32, wherein the administration comprises parenteral administration.

35. The method of any one of claims 30 to 34, wherein the composition is an aqueous composition.

36. The method of any one of claims 31 to 35, wherein the subject has or is suspected of having cancer.

37. The method of claim 36, wherein the cancer is non-small cell lung carcinoma (NSCLC).

38. The method of any one of claims 30 to 37, further comprising administering an immune checkpoint inhibitor (ICI) to the subject.

39. The method of claim 38, wherein the ICI comprises an anti-PD-1 antibody.
